(19) United States
(12) Patent Application Publication
(10) Pub. No.: US 2010/0184090 A1
(43) Pub. Date: Jul. 22, 2010

(54) Method for Generating Stable Cell Lines Expressing High Levels of a Protein of Interest

(75) Inventors: Tara Kristen Crawford McFadd, Cape Elizabeth, ME (US); Meijia Yang, Scituate, MA (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: MERCK SERONO SA, Coinsins, Vaud (CH)

(21) Appl. No.: 12/666,434

(22) PCT Filed: Jul. 16, 2008
(86) PCT No.: PCT/EP08/59313

§ 371 Date: Dec. 23, 2009

Related U.S. Application Data
(60) Provisional application No. 60/961,038, filed on Jul. 17, 2007.

(51) Int. Cl. G01N 33/53; C12P 21/06

(52) U.S. Cl. 435/7.6; 435/71.1

(57) ABSTRACT

This invention relates to industrial production of proteins. More specifically, the invention relates to a method for obtaining cells that stably express a protein of interest, even when cultivated in the absence of selective pressure. DHFR is used as a surrogate marker. The transfected cells are not selected based on resistance to a toxic compound, but based on fluorescence as measured by FACS using fluorescent MTX.

### FIELD OF THE INVENTION

This invention relates to industrial production of proteins. More specifically, the invention relates to a method for obtaining cells that stably express a protein of interest, even when cultivated in the absence of selective pressure. DHFR is used as a surrogate marker. The transfected cells are not selected based on resistance to a toxic compound, but based on fluorescence as measured by FACS using fluorescent MTX.

### BACKGROUND

Introducing heterologous genes into animal host cells and screening for expression of the added genes is a lengthy and complicated process. Typically a number of hurdles have to be overcome: (i) the construction of large expression vectors; (ii) the transfection and selection of clones with stable long-term expression; and (iii) screening for high expression rates of the heterologous protein of interest.

### 1. Selection of Clones Expressing an Heterologous Gene

### 1.1. Screening of Transformants

Selection of the clones having integrated the gene of interest is performed using a selection marker conferring resistance to a selective pressure. Most of the selectable markers confer resistance to an antibiotic such as, e.g., neomycin, kanamycin, hygromycin, gentamycin, chloramphenicol, puromycin, zeocin or bleomycin. When generating cell clones expressing a gene of interest from expression vectors, host cells are typically transfected with a plasmid DNA vector encoding both the protein of interest and the selection marker on the same vector. Quite often the capacity of a plasmid is limited and the selection marker has to be expressed from a second plasmid, which is co-transfected with the plasmid comprising the gene of interest.

Stable transfection by classical methods leads to random integration of the expression vector in the genome of the host cell. Use of selective pressure, e.g. by administrating an antibiotic to the media, will eliminate all cells that did not integrate the vector containing the selection marker providing resistance to the respective antibiotic or selective pressure. If this selection marker is on the same vector as the gene of interest or, if this selection marker is on a second vector and vector comprising the gene of interest was co-integrated, the cells will express both the selection marker and the gene of interest.

### 1.2. Screening of High Producers

Once transformants have been obtained, cells are re-cloned and high producers selected.

One possibility for selecting high producers is to directly quantify expression of the protein of interest using e.g. ELISA. However, high producer cells are usually first screened for high expression of a surrogate marker that can easily be measured. When using a surrogate marker the protein of interest and the surrogate marker are usually located on the same vector and the expression levels of the two proteins are correlated.

Fluorescence-activated cell sorting (FACS) is an easy and convenient method to re-clone cells and to quantify the expression level of a fluorescent surrogate marker such as e.g. the A. victoria or the R. reniformis Green Fluorescent Protein (GFP). Thus selection methods using FACS screening in conjunction with a selective pressure are widely used in the art for selecting high producer cells (see e.g. Gubin et al., 1999; Yoshikawa et al., 2001; DeMaria et al., 2007).

For example, Yoshikawa et al. (2001) discloses an improved method for the selection of highly productive gene-amplified CHO cells by FACS. The cells are selected by resistance to MTX and amplified before screening of high producers cells by FACS using the f-MTX/DHFR system.

The selection pressure is generally removed after selection of transformants in which the nucleic acid encoding POI and the selectable marker has integrated into the genome. High producers are then selected by FACS in the absence of any selective pressure (see e.g. Gubin et al., 1997).

In any event, transformants are first selected in the presence of a selective pressure. Indeed, the attempts to select transfected cells in the absence of selective pressure suggest have proven to be unsuccessful (see e.g. Migliaccio et al. 2000).

Otto et al. (2005) discloses a method for recloning cells wherein no selection pressure is applied. However, this method is intended for recloning of previously transfected cells. In addition, cells are selected by FACS based on expression levels of the ZS Green protein. Since the gene encoding this protein was cloned from a Zoanthus fungus, this may lead to toxicity and/or safety issues during manufacturing.

2. The Limitations Associated with Selective Pressure

Although widely used for screening for high producer cells, the use of a selective pressure is associated with a number of problems.

When removing selective pressure, expression becomes quite often very unstable or even extinguished. Only a small number of initial transformants are thus providing high and stable long-term expression and it is time-consuming to identify these clones in a large population of candidates. Typically, high expressing candidates are isolated and then cultivated in absence of selective pressure. Under these conditions a large proportion of initially selected candidates are eliminated due to their loss of gene of interest expression upon removal of selective pressure. It would thus be advantageous to cultivate the candidates, following an initial period of selection for stable transfection, in absence of selective pressure and only then screen for gene of interest expression.

In addition, selective pressure by the addition of drug is associated with multiple gene copy number events either by random integration or by amplification. Multiple copy number events are associated with gene expression instability, likely due to the loss of copy number over time without selective pressure in media. The result is low titer bio-production fermentation.

Therefore, the finding of a novel and powerful method for isolating high producer cells in which the heterologous protein of interest is stably expressed would be extremely useful in the field of industrial production of therapeutic proteins.

### SUMMARY OF THE INVENTION

The present invention stems from the finding of a method for establishing cell lines stably expressing high levels of a protein of interest. This method, which is based on the use of DHFR as a surrogate marker, is characterized by the fact that it does not require the cells to be selected for drug-resistance. Example 1 discloses such a method according to the invention. This method comprises selecting cells based on the expression of DHFR as determined by fluorescent labeling and FACS analysis, without first selecting transfected cells using a toxic compound. As shown in Examples 2 and 3, this method results in the selection of a cell lines stably expressing high levels of a protein of interest.

Therefore, a first aspect of the invention relates to a method of screening cells for expression of a protein of interest (POI) comprising the steps of:
a) transfecting a cell with:
   (i) a nucleic acid encoding said POI; and
   (ii) a nucleic acid encoding said DHFR;
b) measuring DHFR expression using a fluorescent compound binding to DHFR; and
c) selecting about 0.001% to about 25% of the cells tested in step (b) based on high relative DHFR expression;
   wherein said cells are not selected for resistance to a toxic compound between step (a) and (b).

A second aspect of the invention relates to a method of screening cells for expression of POI comprising the steps of:
a) transfecting a cell with a nucleic acid encoding said POI;
b) measuring POI expression using a fluorescent antibody binding to said POI; and
c) selecting about 0.001% to about 25% of the cells tested in step (b) based on high relative POI expression;
   wherein said cells are not selected for resistance to a toxic compound between step (a) and (b).

A third aspect of the invention relates to a method of obtaining a cell line expressing a POI, said method comprising the step of:
a) screening cells according to any of the above methods of the invention; and
b) establishing a cell line from at least one of said cells.

A fourth aspect of the invention relates to a method of producing a POI, said method comprising the step of:
a) culturing a cell line obtained according to the above method under conditions which permit expression of said POI; and
b) collecting said POI.

A fifth aspect of the invention relates to the use of DHFR either for screening cells for expression of a POI or for obtaining a cell line expressing a POI, characterized in that said cells or cell line are neither selected for resistance to MTX nor for a metabolic advantage in the absence of hypoxanthine and thymidine (HT).

### BRIEF DESCRIPTION OF THE FIGURES

FIG. 1 compares the productivity of a cell line obtained by the method of the present invention and a cell line obtained by conventional drug selection. The Mean Fluorescent Intensity (MFI) is a result of fluorescent-MTX complexed to DHFR. These stable cells were obtained and analysed as described in Examples 1 and 2. PRE-Round 1 refers to the cells before the first FACS-sorting. PRE-Round 2 refers to the cells before the second FACS-sorting. POST-Round 2 refers to the cells after the second FACS-sorting.

FIG. 2 shows the results of a stability study of cells selected using the method of the invention. The studied POI is a variant of the human Chorionic Gonadotropin (hCG). The specific productivity is reported in picograms per cell per day (pcd). The population doubling level (PDL) refers to the cumulative mitosis events. The population doubling time is a measure of the growth.

### DETAILED DESCRIPTION OF THE INVENTION

The present invention stems from the finding of a method for establishing cell lines. Surprisingly, this method does not use drug-resistance to select the stable cell (Example 1). This method allowed isolating cells that expressed similar levels of a protein of interest as a conventional method based on drug-resistance (Example 2). Since the growth media for the recombinant stable cells produced by the FACS method is without drug, there is no pressure for multiple copy number integration or expression of the gene of interest. Therefore, the instability in the absence of drug pressure is not an issue. Complete stable specific productivity has been observed for greater than 50 population doublings (Example 3). Accordingly, the present invention provides a powerful method for isolating cell lines stably expressing a protein of interest.

### 1. The Methods of the Present Invention.

A first aspect of the present invention is directed to a method of screening cells for expression of a protein of interest (POI) comprising the steps of:
a) transfecting a cell with:
   (i) a nucleic acid encoding said POI; and
   (ii) a nucleic acid encoding DHFR;
b) measuring DHFR expression using a fluorescent compound binding to DHFR; and
c) selecting about 0.001% to about 25% of the cells tested in step (b) based on high relative DHFR expression;
   wherein said cells are not selected for resistance to a toxic compound between step (a) and (b).

The term “DHFR” refers to a polypeptide that is a member of the dihydrofolate reductase family (EC 1.5.1.3), and that can catalyze the following enzymatic reaction:
5,6,7,8-tetrahydrofolate+NADP⁺=7,8-dihydrofolate+NADPH

Said nucleic acid encoding DHFR may be of any origin. It may for example be of bacterial origin, e.g., from Escherichia coli (Miller et al., 2005). The DHFR is preferably of eukaryotic origin. More preferably it is of mammalian origin. Most preferably it is of mouse origin (Subramani et al., 1981). In one embodiment, the DHFR gene has been cloned from the same species as the transfected cell. DHFR may correspond to a wild-type DHFR polypeptide or to a mutant thereof, as long as said mutant retains the ability to catalyze the above reaction. Mutant DHFR polypeptides exhibiting modified kinetic parameters are well-known in the art.

The term “transfectinq a cell” should be understood as introducing a recombinant nucleic acid such as e.g. a vector into the cell.

In a population of cells, cells exhibiting “high relative DHFR expression” are those cells which exhibit higher DHFR expression than other cells. For example, cell No. 1 expresses 10 mg/L of DHFR and cell No. 2 expressed 1 mg/l of DHFR. In this example, cell No. 1 exhibits high relative DHFR expression.

As used herein, the term “screening” refers to the testing or examining of a large number of cells for a specific trait.

As used herein, the term “selecting” refers to the choice of some specific cells from a group of cells.

The term “toxic compound” refers to any compound in the presence of which untransfected cells cannot be cultivated because said toxic compound either kills the untransfected cells or inhibits its growth. Examples of such compounds include e.g. MTX, puromycin, neomycin, kanamycin, neomycin, kanamycin, hygromycin, gentamycin chloramphenicol, zeocin and bleomycin.

In a preferred embodiment of the present invention, the cells are neither selected for resistance to a toxic compound nor selected for a metabolic advantage between step (a) and (b).

The term “metabolic advantage” refers to the ability of a transfected cell to grow in the absence of a compound, said compound being mandatory for growth of the untransfected cell. For example, CHO cells comprising the gene encoding the glutamine synthetase (GS) can to grow in the absence of glutamine, and CHO cells comprising the gene encoding DHFR can grow in the absence of thymidine and/or hypoxanthine (HT). If the transfected cell does not comprise (or comprises an inactive) GS or DHFR gene, the transfected cell gains a metabolic advantage that can be selected by cultivating the cells in the absence of glutamine or HT respectively.

Fluorescent compounds binding to DHFR are known in the art and include compounds such as e.g. fluorescently-labelled folate analogues that covalently bind to DHFR. Such fluorescently-labelled folate analogues include fluorescent methotrexate (f-MTX) and fluorescent trimethoprim (f-TMP) (Miller et al., 2005).

The DHFR expression may be measured by any conventional mean for measuring fluorescence such as e.g. a fluorescence microscope, a fluorescence-activated cell sorter (FACS) or the like. It is highly preferred that DHFR expression is measured by FACS.

In order to measure DHFR expression in step (b) of the method of the invention, the cells are incubated in the presence of the fluorescent compounds binding to DHFR. Although such a fluorescent compound binding to DHFR may be toxic for the cells, the duration of such an incubation is too short to kill any cell. As a matter of fact, DHFR-deficient cells need to be cultivated in the presence of MTX or f-MTX for more than 24 hours for any drug selection to take place. Therefore, the incubation step is not a step in which the cells are selected for resistance to the fluorescent compound binding to DHFR.

In a preferred embodiment of the present invention, the cells are incubated in the presence of the fluorescent compounds binding to DHFR for less than 24, 22, 20, 18, 16, 14, 12, 10, 8, 6, 4 or 2 hours. More preferably, the cells are incubated in the presence of the fluorescent compounds binding to DHFR for about 20 or for about 4 hours.

Any cell is suitable for performing the methods of the inventions. The cell may be a primary cell or an established cell line from a wide variety of eukaryotes including plant and animal cells. Preferably, said cell is a eukaryotic cell. More preferably, said cell is a mammalian cell. More preferably, said cell is a CHO cell, a human cell, a mouse cell or a hybridoma. Most preferably, said cell is a CHO-DUKX cell (Urlaub and Chasin, 1980).

In a first embodiment the cell is DHFR-deficient (e.g. CHO-DUKX or CHO-DG44). In the frame of this embodiment, any originally DHFR⁺ cell may be engineered to become DHFR-deficient.

In a second embodiment the cell is DHFR⁺ (i.e., it comprises in its genome a functional endogenous DHFR gene). Indeed, the stable integration of supplemental copies of the DHFR gene can be measured in a cell even when the cell is DHFR⁺ (see e.g. Connors et al. 1988).

In a preferred embodiment the nucleic acid encoding the POI and nucleic acid gene encoding DHFR are located on the same vector being transfected into said cell in step (a). Alternatively, said nucleic acid encoding the POI and said nucleic acid encoding DHFR may be located on separate vectors which are co-transfected into said cell in step (a).

When nucleic acid encoding the POI and nucleic acid gene encoding DHFR are located on the same vector, said vector may comprise at least two promoters, one driving the expression of said nucleic acid encoding the POI, and the other one driving the expression of said nucleic acid encoding DHFR. Alternatively, said nucleic acid encoding the POI may be driven by the same promoter as the nucleic acid encoding DHFR, and said vector comprises either an internal ribosome entry site (IRES) or a 2A sequence between said nucleic acids (de Felipe et al., 2006).

The term “promoter” as used herein refers to a region of DNA that functions to control the transcription of one or more DNA sequences, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured in any of the assays known in the art, e.g. in a reporter assay using DHFR as reporter gene (Seliger and McElroy, 1960; Wood et al., 1984; de Wet et al., 1985), or commercially available from Promega®. An “enhancer region” refers to a region of DNA that functions to increase the transcription of one or more genes. More specifically, the term “enhancer”, as used herein, is a DNA regulatory element that enhances, augments, improves, or ameliorates expression of a gene irrespective of its location and orientation vis-á-vis the gene to be expressed, and may be enhancing, augmenting, improving, or ameliorating expression of more than one promoter.

In a preferred embodiment, the vector of the invention comprises at least one promoter of the murine CMV immediate early region. The promoter may for example be the promoter of the mCMV IE1 gene (the “IE1 promoter”), which is known from, e.g., WO 87/03905. The promoter may also be the promoter of the mCMV IE2 gene (the “IE2 promoter”), the mCMV IE2 gene itself being known from, e.g., Messerle et al. (1991). The IE2 promoter and the IE2 enhancer regions are described in details in WO 2004/081167. Preferably, the vector of the invention comprises at least two promoters of the murine CMV immediate early region. More preferably, the two promoters are the IE1 and the IE2 promoters.

In a preferred embodiment, the vector of the invention comprises at least two promoters of the murine CMV immediate early region, wherein one of them drives the expression of a polypeptide of the invention, and the other one drives the expression of a POI.

In accordance with the present invention, the POI may be any polypeptide for which production is desired. The POI may find use in the field of pharmaceutics, agribusiness or furniture for research laboratories. Preferred proteins of interests find use in the field of pharmaceutics.

For example, the POI may be, e.g., a naturally secreted protein, a normally cytoplasmic protein, a normally transmembrane protein, or a human or a humanized antibody. When the POI is a normally cytoplasmic or a normally transmembrane protein, the protein has preferably been engineered in order to become soluble. The polypeptide of interest may be of any origin. Preferred polypeptides of interest are of human origin.

In preferred embodiments, the POI is selected from the group consisting of chorionic gonadotropin, follicle-stimulating hormone, lutropin-choriogonadotropic hormone, thyroid stimulating hormone, human growth hormone, interferons (e.g., interferon beta-1a, interferon beta-1b), interferon receptors (e.g., interferon gamma receptor), TNF receptors p55 and p75, interleukins (e.g., interleukin-2, interleukin-11), interleukin binding proteins (e.g., interleukin-18 binding protein), anti-CD11a antibodies, erythropoietin, granulocyte colony stimulating factor, granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, Factor VIII, somatropin, bone morphogenetic protein-2, platelet-derived growth factor, hirudin, epoietin, recombinant LFA-3/IgG1 fusion protein, glucocerebrosidase, monoclonal antibodies, and muteins, fragments, soluble forms, functional derivatives, fusion proteins thereof.

Preferably, said monoclonal antibody is directed against a protein selected from the group consisting of CD3 (e.g. OKT3, NI-0401), CD11a (e.g. efalizumab), CD4 (e.g. zanolimumab, TNX-355), CD20 (e.g. ibritumomab tiuxetan, rituximab, tositumomab, ocrelizumab, ofatumumab, IMMU-106, TRU-015, AME-133, GA-101), CD23 (e.g. lumiliximab), CD22 (e.g. epratuzumab), CD25 (e.g. basiliximab, daclizumab), the epidermal growth factor receptor (EGFR) (e.g. panitumumab, cetuximab, zalutumumab, MDX-214), CD30 (e.g MDX-060), the cell surface glycoprotein CD52 (e.g. alemtuzumab), CD80 (e.g. galiximab), the platelet GPIIb/IIIa receptor (e.g. abciximab), TNF alpha (e.g. infliximab, adalimumab, golimumab), the interleukin-6 receptor (e.g. tocilizumab,), carcinoembryonic antigen (CEA) (e.g. 99 mTc-besilesomab), alpha-4/beta-1 integrin (VLA4) (e.g. natalizumab), alpha-5/beta-1 integrin (VLA5) (e.g. volociximab), VEGF (e.g. bevacizumab, ranibizumab), immunoglobulin E (IgE) (e.g. omalizumab), HER-2/neu (e.g. trastuzumab), the prostate specific membrane antigen (PSMA) (e.g. 111 ln-capromab pendetide, MDX-070), CD33 (e.g. gemtuzumab ozogamicin), GM-CSF (e.g. KB002, MT203), GM-CSF receptor (e.g. CAM-3001), EpCAM (e.g. adecatumumab), IFN-gamma (e.g. NI-0501), IFN-alpha (e.g. MEDI-545/MDX-1103), RANKL (e.g. denosumab), hepatocyte growth factor (e.g. AMG 102), IL-15 (e.g. AMG 714), TRAIL (e.g. AMG 655), insulin-like growth factor receptor (e.g. AMG 479, R1507), IL-4 and IL13 (e.g. AMG 317), BAFF/BLyS receptor 3 (BR3) (e.g. CB1), CTLA-4 (e.g. ipilimumab).

Any number of cells may be screened in accordance with the invention. For example, the fluorescence of at least 10, 100, 1,000, 10,000, 100,000, 1,000,000, 5,000,000, 10,000,000, 20,000,000, 30,000,000, 40,000,000, 50,000,000, 60,000,000, 70,000,000, 80,000,000, 90,000,000, 100,000,000 cells may be screened.

Steps (b) (i.e. measuring DHFR expression by fluorescence) and (c) (i.e. selecting the most fluorescent cells) may be iteratively repeated on the population selected at the end of step (c). For example, at least 2, 3, 4, 5 or 10 iterations may be carried out. This may be done with or without changing conditions in between the selection steps. Changing conditions may include e.g. varying culture conditions such as media components or physico-chemical parameters.

In a preferred embodiment, the cell selected after the last iteration of step (c) exhibits stable expression of the POI in the absence of any drug selection for at least 10, 20, 30, 45 or 50 population doubling levels (PDL).

In a preferred embodiment, the cell selected after the last iteration of step (c) does not loose more than 20%, 15%, 10%, 5% or 1% of its specific productivity (pcd) after 15 PDL. Preferably, said cell not loose more that 20%, 15%, 10%, 5% or 1% of its specific productivity (pcd) after 50 PDL. More preferably, said cell not loose more that 10% of its specific productivity (pcd) after 50 PDL. Most preferably, said cell does not loose any of its specific productivity (pcd) after 50 PDL.

In one embodiment of the invention, the cells selected at the end of step (c) are subjected to a further screening comprising the steps of (i) transfecting a cell with a nucleic acid encoding said POI; (ii) measuring POI expression using a fluorescent antibody binding to said POI; and (iii) selecting about 0.001% to about 25% of the cells tested in step (b) based on high relative POI expression.

After the last selection based on fluorescence (the last iteration of step c), the expression level of the POI in said selected cells may further be measured (step d).

The expression level of the POI may be measured by any method known in the art, such as e.g. ELISA, FACS, Northern blot or RT-PCR.

Then, the about 0.001% to about 25% of the cells tested in step (d) may be selected based on high relative expression of the POI (step e). For example, about 0.001%, 0.005%, 0.01%, 0.5%, 1%, 1%, 5%, 2%, 3%, 4%, 5% to about 15%, 18%, 20% or 25% of the cells exhibiting high relative expression of the POI may be selected.

A further aspect of the invention pertains to a method of obtaining a cell line expressing a POI, said method comprising the steps of:
a) screening cells according to the above method; and
b) establishing a cell line from said cells.

As used herein, a “cell line” refers to one specific type of cell that can grow in a laboratory. A cell line can usually be grown in a permanently established cell culture, and will proliferate indefinitely given appropriate fresh medium and space. Methods of establishing cell lines from isolated cells are well-known by those of skill in the art. In a preferred embodiment said cell line is a stable cell line, i.e., a cell line that does not loose more that 20%, 15%, 10%, 5% or 1% of its specific productivity (pcd) in the absence of any drug selection for at least 10, 20, 30, 45 or 50 population doubling levels (PDL). Preferably, said cell line does not loose more that 10% of its specific productivity (pcd) after 50 PDL. Most preferably, said cell line does not loose any of its specific productivity (pcd) after 50 PDL.

Another aspect relates to a method of producing a POI, said method comprising the step of:
a) culturing a cell line obtained as described above under conditions which permit expression of said POI; and
b) collecting said POI.

Conditions which permit expression of the POI can easily be established by one of skill in the art by standard methods. For example, the conditions disclosed in Example 3.3.1 may be used.

In a preferred embodiment, the above method of producing a POI further comprises the step of purifying said POI. The purification may be made by any technique well-known by those of skill in the art. In the case of a POI for use in the field of pharmaceutics, the POI is preferably formulated into a pharmaceutical composition. A further aspect of the invention pertains to the use of DHFR for screening cells for expression of a protein of interest (POI), characterized in that said cells are neither selected for resistance to MTX nor for a metabolic advantage in the absence of hypoxanthine and thymidine (HT).

Still another aspect of the invention pertains to the use of DHFR for obtaining a cell line expressing a POI, characterized in that said cell line is neither selected for resistance to MTX nor for a metabolic advantage in the absence of HT. Preferably, said cell line stably expresses the POI.

Alternatively, the fluorescence of the transfected cell can be measured using a fluorescent antibody binding to the POI instead of being measured using a fluorescent compound binding to DHFR. Typically, such a method of screening cells for expression of a protein of interest (POI) comprises the steps of:
a) transfecting a cell with a nucleic acid encoding said POI;
b) measuring POI expression using an antibody binding to said POI; and
c) selecting about 0.001% to about 25% of the cells tested in step (b) based on high relative POI expression;
   wherein said cells are not selected for resistance to a toxic compound between step (a) and (b).

All other steps are identical to the steps in the method above wherein DHFR is used.

### 2. Advantages of the Present Invention Over the Prior Art.

This method provides a FACS based selection process for obtaining stable cells that completely eliminates the need for drug-resistance selection.

In this method, the FACS screening process for high producer cells is incorporated into the selection process for stable cells. This screening method has thus the advantage of comprising a reduced number of steps.

This process further allows selecting stable cell lines in the absence of selective pressure. It has indeed been shown that expression of the POI by the selected cells is stable without drug selection pressure for over fifty population doublings. This is extremely advantageous when industrially producing a protein.

In addition, the cells are never in the presence of drug to maintain stability of gene expression and need not be for the manufacturing process.

Finally, DHFR is a naturally-occurring protein that is naturally present in eukaryotic cells such as e.g. CHO cells. In other words, the cell line obtained using the methods of the present invention only expresses two recombinant proteins: the recombinant POI and the recombinant DHFR protein, the gene of which is in any case naturally present in said cell. This is a major difference with a cell line that comprises markers of bacterial and/or cyanobacterial such as e.g. GFP or ZS-Green. In particular, the expression of DHFR in the cell line for manufacturing the protein of interest does not lead to toxicity issues as is the case when using e.g. GFP or ZS-Green as a surrogate marker. The cell line obtained using the methods of the present invention is therefore safer for manufacturing purposes.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Example 1

### Protocol

### 1.1. Cells, Media and Plasmids

A Chinese hamster ovary cell line derived from CHO-DUKX-B11 was developed for stable expression of the proteins of interest. Prior to transfection, host cells were adapted to serum-free high-density suspension growth in a chemically defined medium (further referred to as the “Growth Media”) that was derived from PROCHO-5 media (Bio-Whittaker/Cambrex, East Rutherford, N.J.) and that was supplemented with 2% sodium hypoxanthine and thymidine (HT). All reagents were furnished from Invitrogen Corp., Carlsbad, Calif., unless noted otherwise. Cells for transfection were cultured in disposable shake flasks containing the afore-mentioned growth media, shaking at 125 rpm, in a 37° C., 5% CO₂ incubator.

The transfections consisted of the applied combination of linearized plasmid DNA coding alpha and beta subunits of an inactive version of the heterodimeric hormone, human chorionic gonadatropin (hCG). The expression vector (referred to as D-alpha) contained the gene of interest N-terminally fused to the human Growth Hormone signal sequence and promoted by the metallothionien 1 promoter (MTT-1). It further contained the wild-type murine DHFR gene promoted by the sv-40 early promoter and a gene for ampicillin resistance. The D-alpha vector has been described in more detail in Kelton et. al. (1992).

### 1.2. Transfection

90 μl of Lipofectamine reagent (DMRIE-C) and DNA (15 μg total, 1:1 volumetric ratio of subunits) were separately mixed with 1.5 mL growth media and the mixtures were incubated for 10 min at room temperature, then combined to allow DNA-lipid complex formation through an additional 30 minutes incubation at room temperature. The 20 million host cells were harvested for transfection by centrifugation at 800 rpm for 5 minutes, and cell pellets were resuspended in 3 mL Growth Medium in 25 mL size T-flasks. The lipid/DNA mixture was added to the cells and incubated stationary at 37° C. with 5% CO₂. After four hours, growth media was added to reach 40 mL culture volume, and the cells were transferred to a 250 mL shake flask. The cell cultures were maintained by shaking at 125 rpm, in a 37° C., 5% CO₂ environment.

### 1.3. First FACS-Sorting

Forty-eight hours post transfection, the cells were counted and 20 million cells were collected and centrifuged as previously described. The cell pellets were resuspended in 20 mL of PROCHO-5+supplemented with 4 mM L-glutamine and 5 μM fluorescein MTX (f-MTX) and incubated overnight by shaking in the dark at 125 rpm, in a 37 degree C., 5% CO2 environment.

The following day, the labeled cells were pelleted by centrifugation and underwent two rounds of washing with 5 mL of cold Phosphate Buffered Saline (PBS)+0.5% Pluronic Acid +25 μM Hepes Buffer. The cells were then re-suspended in 2 mL of wash buffer and kept on ice prior to evaluation of fluorescence by flow cytometry using a FACS Aria (BD Biosciences, San Jose, Calif.) set for flourescein isothiocyanate (FITC) excitation by the 488 nM line of the argon lasor. The sheath pressure was set to low at 25 psi and the nozzle size was 100 micron.

The FACS gating process consisted of a first gate (selection of population one, referred to as P1), based on light scatter events representing the physical parameters of the cell; forward and side scatter (FSC-H/SSC-H dot plot). The parameters were set according to the supplier's manual (BD FACS Aria Operators Manual, edition 336951 Rev A., August 2003). P1 was then further gated into quadrants on a FSC-W/FITC-A dot plot. Quadrant one (Q1) contained the positive fluorescent cells (indicating DHFR expression due to vector insertion) with low FSC-W (indicating single cell evaluation).

Host cells were loaded onto the FACSAria and voltage settings were optimized: Scatter Events ˜100,000 and mean fluorescent intensity <200. Drop delay was determined using Accudrop technology (BD FACS Aria Operators Manual). Host cells undergone the single cell gate (P1) and the population was subsequently displayed in the quadrant gate (Q1-Q4). The minimal threshold for the x-axis quadrant gate was set as a baseline for fluorescence. There were no background events in Q1, which corresponded to the desired gate for sorting. To select the cells expressing the gene of interest, the transfected pool of cells were then loaded on the FACS Aria and gated as previously described. The analysis of the desired population, Q1, was compatible with a separation of means (FITC) by a factor of 19.1 (as compared to P1), which constituted 3.1% of the total. Single cells from the Q1 gate were sorted and pooled into a collection tube containing 2 mL of Growth Media supplemented with 1% dialyzed fetal bovine serum and 2× penicillin streptomycin to prevent bacterial contamination (the “Post-Sort Media”). Cells were pelleted by centrifugation and re-suspended in 5 mL Post-Sort Media in a stationary T25 flask to confluency. The culture was then trypsinized and scaled up to shake flasks and cultured in suspension as previously described.

The sorted population “Q1” was cultured in Post-Sort Media for approximately 14 days. Prior to the second round of sorting, an expression analysis was performed. For characterizing expression of selected pool, conditioned media was generated by seeding 1 million cells/mL in 10 ml selection media and cultured in a disposable shake flask for 24 hours at 37° C. The conditioned media was cleared by centrifugation for 5 minutes at 800 rpm, and the supernatant was stored at 4 degrees for analytical characterization. Protein expression was quantified by using DSL hCG ELISA and following manufacturer's protocol with kit standards (Diagnostic Systems Laboratories, Webster, Tex.).

Specific productivity was calculated using the following equation:

Qₛₚ(p/c/d)=(P₂−P₁)×ln(Nₜ/Nₒ)/(T₂−T₁)(Nₜ−Nₒ)

P1: InitialTiter (in picograms)
P2: Final Titer (in picograms)
Nₜ: Final Population Size (number of cells)
Nₒ: Initial Population Size (number of cells)
T1: Time of Initiation (day)
T2: Time of harvest (day)

The average specific productivity of the cells prior to the second sort was 0.2 pcd.

### 1.4. Second FACS-Sorting

To isolate the highest expressing cells, the population of cells generated from the first sort (Q1) were scaled up, labeled with f-MTX for 4 hours, and put through an additional round of evaluation and sorting as previously described. The analysis of the sorted population, Q1-Q1, was compatible with a separation of means (FITC) by a factor of 8.4 (as compared to P1), which constituted 1.3% of the total.

Cells were sorted into a stationary T25 flask with 5 mL Post-Sort Media minus HT and subsequently scaled up to shake flasks and cultured as previously described. An expression analysis was performed on the pool of cells following the final sort, resulting in an average specific cellular productivity of 3.2 pcd.

### Example 2

### Mean Fluorescence Intensity (MFI) Comparison of FACS-Selected and Drug-Selected Cells

As a control, a drug selected cell line was established as follows: forty-eight hours post-transfection, the cells were transferred into selection medium (0.5 μM Methotrexate in PROCHO-5 media supplemented with 4 mM L-glutamine). Every two to three days, the cells were passaged by centrifugation and re-suspension in fresh selection media to yield a final density of 5×10⁵ cell/mL. After approximately three weeks the cells showed signs of growth recovery. The CHO pool was considered stable when the growth rate was constant and the viability was >90%. The cells were labeled and evaluated for fluorescence by FACS at the various processing points (pre-sort one, pre-sort two, and post sort-two for FACS selected cell line; after selection process for drug-selected cell line) as previously described. The single cell gate (P1) was displayed on a FITC-A histogram.

A clear shift in MFI is observed as the transfected cells were processed through multiple rounds of FACS-selection (1a-1c).

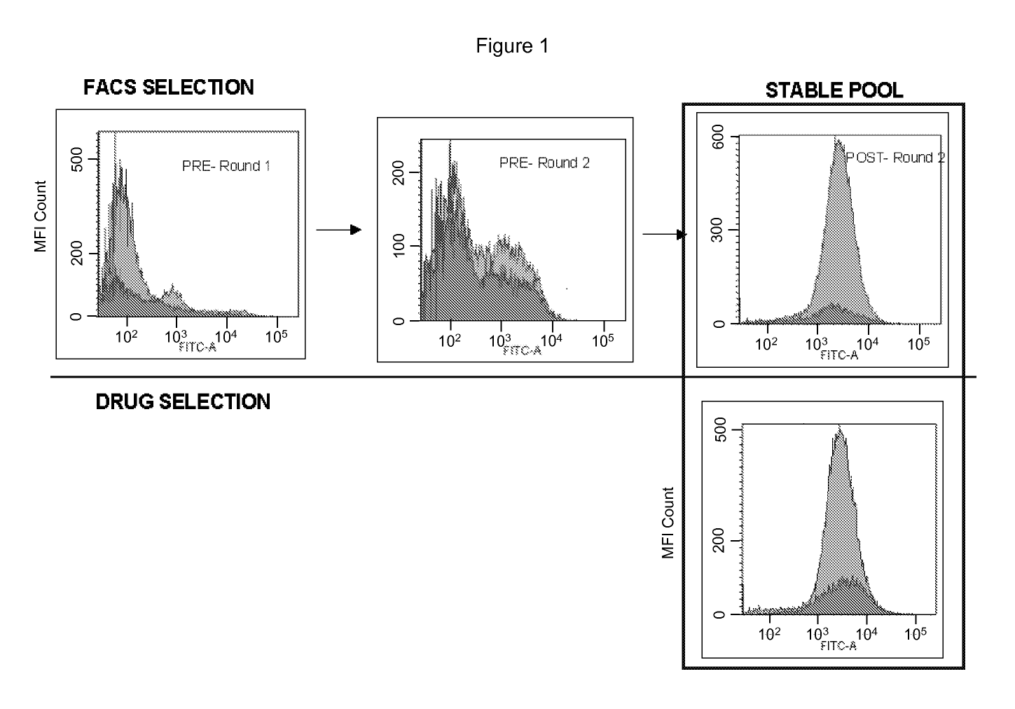

Stable CHO cells generated by FACS-selection (1c) and grown in culture media without drug selection pressure show a similar profile to the drug selected cell line (1d) grown in the presence of 0.5 uM MTX. The result shown in FIG. 1 shows that the method of the invention allowed obtaining high-producing recombinant cell lines.

### Example 3

### Stability Evaluation

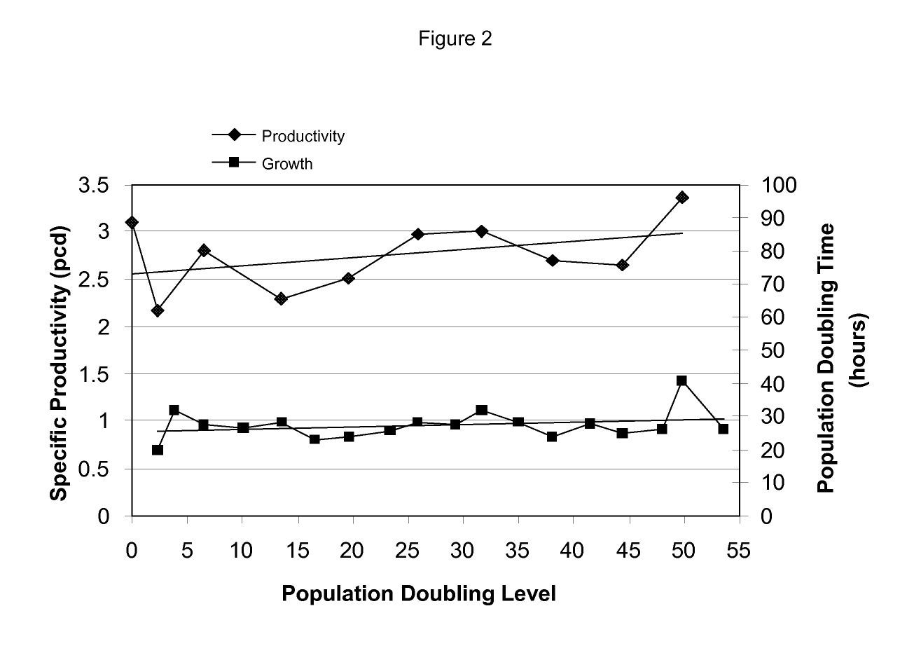

The cells were cultured for 50 population doubling lengths in post sort media (without drug selection) to address the stability of gene expression from the FACS-selected stable cell line. Weekly expression analysis was conducted to determine specific cellular productivity. The result are shown in FIG. 2.

The cells stably expressed, without any loss in average specific productivity, the hCG protein after 50 Population Doubling Lengths (PDL) from the final sort. The cells maintained a consistent growth rate with an average doubling time of 27 hours.

In conclusion, the method of the invention results in the selection of a stable, high-producing recombinant cell line that can be used for the research or manufacturing of proteins.

### REFERENCES

Connors, R. W., Sweet, R. W., Noveral, J. P., Pfarr, D. S., Trill, J. J., Shebuski, R. J., Berkowitz, B. A., Williams, D., Franklin, S., Reff, M. E. (1988) DHFR coamplification of t-PA in DHFR+ bovine endothelial cells: In vitro characterization of the purified serine protease. DNA 7, 651-661
de Felipe, P., Luke, G. A., Hughes, L. E., Gani, D., Halpin, C., and Ryan, M. D. (2006). E

unum pluribus: multiple proteins from a self-processing polyprotein. Trends Biotechnol. 24, 68-75.
de Wet, J. R., Wood, K. V., Helinski, D. R., and DeLuca, M. (1985). Cloning of firefly luciferase cDNA and the expression of active luciferase in Escherichia coli. Proc Natl Acad Sci USA 82, 7870-7873.
DeMaria, C. T., Cairns, V., Schwarz, C., Zhang, J., Guerin, M., Zuena, E., Estes, S., and Karey, K. P. (2007). Accelerated clone selection for recombinant CHO CELLS using a FACS-based high-throughput screen. Biotechnol. Prog. 23, 465-472.
Gubin, A. N., Koduru, S., Njoroge, J. M., Bhatnagar, R., and Miller, J. L. (1999). Stable expression of green fluorescent protein after liposomal transfection of K562 cells without selective growth conditions. Biotechniques 27, 1162-1170.
Gubin, A. N., Reddy, B., Njoroge, J. M., and Miller, J. L. (1997). Long-term, stable expression of green fluorescent protein in mammalian cells. Biochem. Biophys. Res. Commun. 236, 347-350.
Kelton, C. A., Cheng, S. V. Y., Nugent, N. P., Schweickhardt, R. L., Rosenthal, J. L., Overton, S. A., Wands, G. D., Kuzeja, J. B., Luchette, C. A., and Chappel, S. C. (1992) The cloning of the human follicle stimulating hormone receptor and its expression in COS-7, CHO, and Y-1 cells. Molecular and Cellular Endocrinology, 89, 141-151.
Messerle, M., Keil, G. M., and Koszinowski, U. H. (1991). Structure and expression of murine cytomegalovirus immediate-early gene 2. J. Virol. 65, 1638-1643.
Migliaccio, A., R., Bengra, C., Ling, J., Pi, W., Li, C., Zeng, S., Keskintepe, M., Whitney, B., Sanchez, M., Migliaccio, G., and Tuan, D. (2000). Stable and unstable transgene integration sites in the human genome: extinction of the Green Fluorescent Protein transgene in K562 cells. Gene. 256, 197-214.
Miller, L. W., Cai, Y., Sheetz, M. P., and Cornish, V. W. (2005). In vivo protein labeling with trimethoprim conjugates: a flexible chemical tag. Nat. Methods 2, 255-257.
Otto, R., Enenkel, B., Fieder, J., and Krieg, T. (2005) Method for recloning production cells. US 2005/0059146 A1.
Seliger, H. H. and McElroy, W. D. (1960). Spectral emission and quantum yield of firefly bioluminescence. Arch. Biochem. Biophys. 88, 136-141.
Subramani, S., Mulligan, R., and Berg, P. (1981) Expression of the Mouse Dihydrofolate Reductase Complementary Deoxyribonucleic Acid in Simian Virus 40 Vectors. Mol. Cell Biol. 1, 854-864.
Urlaub, G. and Chasin, L. A. (1980). Isolation of Chinese hamster cell mutants deficient in dihydrofolate reductase activity. Proc. Natl. Acad. Sci. U.S. A 77, 4216-4220.
Wood, K. V., de Wet, J. R., Dewji, N., and DeLuca, M. (1984). Synthesis of active firefly luciferase by in vitro translation of RNA obtained from adult lanterns. Biochem Biophys. Res. Commun. 124, 592-596.
Yoshikawa, T., Nakanishi, F., Ogura, Y., Oi, D., Omasa, T., Katakura, Y., Kishimoto, M., and Suga, K. I. (2001). Flow cytometry: an improved method for the selection of highly productive gene-amplified CHO cells using flow cytometry. Biotechnol. Bioeng. 74, 435-442.

1-18. (canceled)

19. A method of screening cells for expression of a protein of interest (POI) comprising the steps of:
(a) transfecting a cell with:
(a) a nucleic acid encoding said POI; and
(ii) a nucleic acid encoding dihydrofolate reductase (DHFR);
(b) measuring DHFR expression using a fluorescent compound binding to DHFR; and
(c) selecting about 0.001% to about 25% of the cells tested in step (b) based on high relative DHFR expression;
wherein said cells are not selected for resistance to a toxic compound between step (a) and (b).

20. The method of claim 19, wherein said DHFR expression is measured by a fluorescence-activated cell sorter (FACS).

21. The method of claim 19, wherein said fluorescent compound binding to DHFR is fluorescent methotrexate (f-MTX) or fluorescent trimethoprim (f-TMP).

22. The method of claim 19, wherein said cell is selected from the group consisting of a human cell, a CHO cell, a murine cell and a hybridoma.

23. The method of claim 19, wherein said cell is a DHFR-deficient cell.

24. The method of claim 23, wherein said cell is a CHO-DUKX cell.

25. The method of claim 19, wherein said nucleic acid encoding the POI and nucleic acid gene encoding DHFR are located on the same vector being transfected into said cell in step (a).

26. The method of claim 25, wherein said vector comprises at least two promoters, one driving the expression of said nucleic acid encoding the POI, and the other one driving the expression of said nucleic acid encoding DHFR.

27. The method of claim 25, wherein said nucleic acid encoding the POI is driven by the same promoter as the nucleic acid encoding DHFR, and wherein said vector comprises either an internal ribosome entry site (IRES) or a 2A sequence located between said nucleic acids.

28. The method of claim 19, wherein steps (b) and (c) are repeated at least 2 times.

29. The method of claim 19, further comprising the step of:
(d) measuring the expression level of the POI in the cells selected at the end of the last step (c).

30. The method of claim 29, further comprising the step of:
(e) selecting about 0.001% to about 25% of the cells tested in step (d) based on high relative expression of the POI.

31. A method of obtaining a cell line expressing a POI, said method comprising the step of:
a) screening cells according to the method of any of the preceding claims; and
b) establishing a cell line from at least one of said cells.

32. A method of producing a POI, said method comprising the step of:
a) culturing a cell line obtained according to the method of claim 31 under conditions which permit expression of said POI; and
b) collecting said POI.

33. The method of claim 32, further comprising the step of purifying said POI.

34. The method of claim 33, further comprising the step of formulating said POI into a pharmaceutical composition.

35. A method of screening cells for expression of a protein of interest (POI) comprising the steps of
a) transfecting a cell with a nucleic acid encoding said POI;
b) measuring POI expression using a fluorescent antibody binding to said POI; and
c) selecting about 0.001% to about 25% of the cells tested in step (b) based on high relative POI expression;
wherein said cells are not selected for resistance to a toxic compound between step (a) and (b).
